(19) United States
(12) Patent Application Publication
(10) Pub. No.: US 2026/0048058 A1
(43) Pub. Date: Feb. 19, 2026

(54) VALACYCLOVIR AND CELECOXIB FOR THE TREATMENT OF ALZHEIMER'S DISEASE AND COVID-19

(71) Applicant: Dogwood Thepapeutics, Inc., Alpharetta, GA (US)

(72) Inventor: William Langley PRIDGEN, Tuscaloosa, AL (US)

(21) Appl. No.: 19/101,137

(22) PCT Filed: Sep. 15, 2023
(86) PCT No.: PCT/US2023/032842

§ 371 Date: Feb. 4, 2025

Related U.S. Application Data
(60) Provisional application No. 63/407,224, filed on Sep. 16, 2022.
(60) Provisional application No. 63/524,391, filed on Jun. 30, 2023.

(51) Int. Cl. A61K 31/522; A61K 31/415; A61P 25/28; A61P 43/00

(52) U.S. Cl. CPC A61K 31/522; A61K 31/415; A61P 25/28; A61P 43/00

(57) ABSTRACT

The present disclosure relates to methods of treating Alzheimer's disease, diseases and/or conditions associated with Covid-19 infection, including long COVID, a post-acute infection syndrome, or symptoms of orthostatic intolerance comprising administration of a therapeutically-effective combination of a COX-2 inhibitor and an antiviral compound.

### CROSS-REFERENCE TO RELATED APPLICATIONS

This application claims the benefit and priority to U.S. Provisional Patent Application Nos. 63/407,224, filed on 16 Sep. 2022, and 63/524,391, filed on 30 Jun. 2023. The entire disclosure of the applications identified in this paragraph are incorporated herein by reference.

### FIELD

The present disclosure relates to methods of treating human diseases including treating Alzheimer's disease, treating or ameliorating diseases or conditions associated with SARS-CoV2 infections, and/or treating or ameliorating a post-acute infection syndrome.

### BACKGROUND

Infection with SARS-CoV2 was first reported around November, 2019. Since then, the virus has spread across the world causing an illness generally referred to as “COVID-19.” Estimates suggest that between 14% and 19% of people with COVID-19 develop long-term symptoms. [Logue, et al., J. Am. Med. Assoc. (2020); Garrigues et al., J. Infect. 81(6):e4-e6 (2020).] The chronic condition is commonly known as “long COVID” or “long haul COVID.” Evidence suggests that long COVID is significantly underreported. There are currently no established, FDA-approved treatments for long COVID.

Alzheimer's disease and long COVID may share similar viral pathogeneses and symptoms with fibromyalgia and other somatic symptom disorders. In particular, Alzheimer's disease, long COVID, and fibromyalgia present with fatigue, cognitive dysfunction (“brain fog”), sleep disorders, muscle weakness, pain, and mood disorders. Herpes virus persistent infection is theorized as a possible cause of fibromyalgia and Alzheimer's disease. Many of the symptoms of long COVID overlap with fibromyalgia and somatic symptom disorders. The HSV-1 downregulates proinflammatory cytokines, such as IL-1, IL-6, and TNF-α. These cytokines are correlated with the development and severity of fibromyalgia and might play a similar role in Alzheimer's disease, and long COVID.

Post-acute infection syndrome (PAIS) can be a sequelae of infection with a virus or bacteria. PAIS can lead to severe chronic fatigue, exercise intolerance, autonomic dysfunction, unrefreshing sleep, memory impairment, and mood changes. See Choutka, et al. (Nat Med, May 2022; 28: 911-923). In some cases, PAIS can lead to complex multisystem disorders, such as ME/CFS, fibromyalgia, and irritable bowel syndrome. See Komaroff, et al. (Front Med, January 2021; 7).

Combinations of an antiviral compound (e.g., famciclovir or valacyclovir) and a COX-2 inhibitor (e.g., celecoxib) for the treatment of functional somatic syndromes (FSS) and fibromyalgia appear to possess viral inhibitory synergy and are described in U.S. Pat. No. 8,809,351.

### SUMMARY

In one embodiment, there is provided a method for treating a subject susceptible to or afflicted with Alzheimer's disease, the method comprising administering to the subject a therapeutically-effective combination of a COX-2 inhibitor (which may also inhibit COX-1) and an antiviral compound.

In one embodiment, there is provided a method for treating a subject susceptible to or afflicted with COVID-19, including long COVID-19, the method comprising administering to the subject a therapeutically-effective combination of a COX-2 inhibitor and an antiviral compound.

In one embodiment, there is provided a method for treating a subject susceptible to or afflicted with PAIS, the method comprising administering to the subject a therapeutically-effective combination of a COX-2 inhibitor and an antiviral compound.

In one embodiment, there is provided a method for treating a symptoms of orthostatic intolerance in a subject suffering from PAIS, the method comprising administering to the subject a therapeutically-effective combination of a COX-2 inhibitor and an antiviral compound.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

FIG. 1 depicts the PROMIS fatigue T score change from baseline for both the combination and SOC control at Week 14.

FIG. 2 depicts the NRS fatigue score change from baseline for both the combination and SOC control at Week 14.

FIG. 3 depicts the NRS pain scale change from baseline for both the combination and SOC control at Week 14.

FIG. 4 shows the PGIC responder rate at Week 14.

FIG. 5 depicts the OISA, OISAS change from baseline for both the combination and SOC control at Week 14.

FIG. 6 depicts the OIDAS change from baseline for both the combination and SOC control at Week 14.

### DETAILED DESCRIPTION

The following description is merely exemplary in nature and is not intended to limit the present disclosure, application, or uses.

### A. Definitions

The term “pharmaceutically acceptable” means suitable for use in pharmaceutical preparations, generally considered as safe for such use, officially approved by a regulatory agency of a national or state government for such use, or being listed in the U. S. Pharmacopoeia or other generally recognized pharmacopoeia for use in animals, and more particularly in humans.

The term “therapeutically-effective amount” refers to an amount of a compound that, when administered to a subject for treating a disease, is sufficient to effect treatment for the disease. “Therapeutically effective amount” can vary depending on the compound, the disease and its severity, the age, the weight, etc. of the subject to be treated.

The term “COX-2 inhibitor” refers to a cyclooxygenase-2 inhibitor, which is any pharmaceutically acceptable compound that inhibits the enzyme cyclooxygenase-2.

The term “COX-1 inhibitor” refers to a cyclooxygenase-1 inhibitor, which is any pharmaceutically acceptable compound that inhibits the enzyme cyclooxygenase-1.

The term “HSV-1” refers to herpes simplex virus-1.

The terms “prevent,” “prevention,” or “preventing” refer to either preventing the onset of preclinically evident condition altogether or preventing the onset of a preclinical evident stage of the condition in a subject. Prevention includes, but is not limited to, prophylactic treatment of a subject at risk of developing a condition.

The term “treat” (and corresponding terms “treatment” and “treating”) includes palliative, restorative, and preventative treatment of a subject. The term “palliative treatment” refers to treatment that eases or reduces the effect or intensity of a condition in a subject without curing the condition. The term “preventative treatment” (and the corresponding term “prophylactic treatment”) refers to treatment that prevents the occurrence of a condition in a subject. The term “restorative treatment” refers to treatment that halts the progression of, reduces the pathologic manifestations of, or entirely eliminates a condition in a subject.

Alzheimer's disease (AD) is a medical disorder characterized by progressive neurodegeration, severe cognitive decline, short-term memory loss, impaired speech, and an inability to perform common daily tasks.

COVID-19 refers to the severe acute respiratory syndrome caused by infection with the RNA virus SARS-CoV-2. Long COVID, also known as “long haul COVID,” refers to the condition wherein subjects experience the effects of the infection for an extended period of time (e.g., months or years) following recovery from the initial infection with SARS-CoV-2. Such long-term effects include, but are not limited to severe chronic fatigue that is not due to ongoing exertion or a medical condition and that significantly interferes with daily activities.

The term “CFS” refers to chronic fatigue syndrome. In some embodiments, the CFS is myalgic encephalomyelitis/CFS (ME/CFS)

The term “cognitive dysfunction”, also referred to as “brain fog”, “mental fog”, or “impaired cognition”, refers to the loss or impairment of intellectual function (such as thinking, remembering, or reasoning) of sufficient severity to interfere with daily functioning.

The terms “famcyclovir” and “famciclovir” refer to the same antiviral compound.

The terms “valacyclovir” and “valaciclovir” refer to the same antiviral compound.

The terms “acyclovir” and “aciclovir” refer to the same antiviral compound.

The term “QD” refers to once a day.

The term “BID” refers to two times a day.

The term “TID” refers to three times a day.

The term “QID” refers to four times a day.

The term PO refers to oral administration.

The term “Likert Survey” (and the corresponding term “Likert Scale”) refers to a questionnaire which asks subjects the extent to which they agree or disagree with a statement, using a five-point scale.

The term “combination therapy” (or “co-therapy”), in defining use of an antiviral compound and a COX-2 inhibitor, as described herein, is intended to embrace administration of each agent in a sequential manner in a regimen that will provide beneficial effects of the drug combination, and is intended as well to embrace co-administration of these agents in a substantially simultaneous manner, such as by oral ingestion of a single capsule having a fixed ratio of these active agents or ingestion of multiple, separate capsules for each agent. “Combination therapy” will also include simultaneous or sequential administration by intravenous, intramuscular, or other parenteral routes into the body, including direct absorption through mucous membrane tissues, as found in the sinus passages. Sequential administration also includes drug combination where the individual elements may be administered at different times and/or by different routes but which act in combination to provide a beneficial effect. It is expected that this combination therapy of an antiviral compound and a COX-2 inhibitor will result in co-action of the antiviral compound and the COX-2 inhibitor, providing a pharmacokinetic interaction, or a pharmacodynamic interaction, or both, where the compounds are administered either simultaneously or sequentially, to permit such co-action.

### B. Clinical Observations

The present invention is to be understood as embracing treatment of Alzheimer's disease and COVID-19, including long COVID. U.S. Pat. No. 8,809,351 reports that a combination of a COX-2 inhibitor and an antiviral agent (e.g., famciclovir and celecoxib or valacyclovir and celecoxib) are useful for treating functional somatic syndromes (FSS), including but not limited to fibromyalgia, as well as pain and associated functional symptoms associated with fibromyalgia. Patients with fibromyalgia were observed to display a variety of symptoms, including but not limited to fatigue, insomnia, depression, allodynia, headaches, irritable bowel syndrome, sensitivity to light, numbness and anxiety. Stress often exacerbates the symptoms. While the etiology and pathogenesis of FSS is not clearly understood, a combination of interactions among external stressors, neurotransmitters, hormones, the immune system, and the sympathetic nervous system, appear to be involved. In particular, Herpes virus is hypothesized to play a major role in fibromyalgia and related functional somatic syndromes.

Herpes viruses including HSV-1 and HSV-2 are unique because after the initial infection they may remain dormant in tissue until conditions are sufficient for a reactivation. Physiologic stressors associated with this reactivation process may initiate the synthesis and release of host peptides and hormones of the sympathetic nervous system and the hypothalamic-pituitary-adrenal axis. Most viruses in this class infrequently reactivate. Both HSV-1 and HSV-2 reactivate often enough to create the milieu needed for development of a chronic debilitating illness. HSV-2 cycles only once or twice a year and is an unlikely candidate for chronic illness. HSV-1, however, cycles frequently enough, on average 4 times a year, and as often as monthly, to result in a slowly developing debilitating illnesses. Without being bound by theory, U.S. Pat. No. 8,809,351 theorizes that after many reactivations, a neuronal cell body dies due to apoptosis and that the ganglion undergoes destruction in the regions governing the first inoculation site.

The existence of Herpes virus in one or more ganglia may directly or indirectly affect the central nervous system (CNS), hypothalamic pituitary axes (HPA) and immune system. Dysregulation of pain processing within the CNS may lead to an amplified perception of pain and other sensory stimuli. This phenomenon, often referred to as central sensitization or augmentation, results from changes in the properties of neurons in the CNS where pain is no longer coupled, as acute nociceptive pain is, to the presence, intensity, or duration of noxious peripheral stimuli. Neurotransmitters such as glutamate, Substance P, serotonin, norepinephrine, dopamine, brain-derived neurotrophic factor (BDNF), and gamma aminobutyric acid (GABA) are activated in chronic pain and depression. Substance P, cerebrospinal fluid levels and serum concentration of BDNF have been consistently higher in patients with fibromyalgia compared with controls. Patients with fibromyalgia also have an abnormal dopamine response to pain. Recent data suggest a putative role of pro-inflammatory cytokines, including interleukin-1-beta, tumor necrosis factor-alpha (TNFα), IL-6 and IL-8, in the pathogenesis of fibromyalgia and the modulation of symptoms [M. DiFranco, et al., Ann. N. Y. Acad. Sci. 1193(1), 84-90(2010)]. Hence pain, as the defining characteristic of fibromyalgia, is not due to tissue damage or inflammation and is thus fundamentally different from rheumatic disorders and many other pain conditions as these conditions cause inflammation in the joints and tissues. Herpes virus has developed various immune evasion mechanisms which prove to be a particular challenge for the immune system. Cytokines and cytokine-induced genes are important for the ability of any organism to raise an antiviral response. Understanding the immune mediators and their possible role in fibromyalgia may be the most daunting obstacles in understanding the disorder. Gur and Oktayoglu [A. Gur and P. Oktayoglu, Curr. Pain Headache Rep. 12(3), 175-181(2008)] explain how cytokines related to acute or repetitive tissue injuries may be responsible for long-term activation of spinal cord glia and dorsal horn neurons, thus resulting in central sensitization. The immune system responds to stressors by causing certain immune cells to secrete the pro-inflammatory cytokines IL-1 and IL-6. Both cytokines are involved in inflammation, and IL-6 is thought to worsen the symptoms of autoimmune disease and fibromyalgia [L. Vanderhaeghe, Total Health 23, 34-35(2001)]. IL-1 and IL-6 are both correlated with the development and severity of Alzheimer's disease. [Su, et al., Neurosci. Bull., 32(5), 469-480 (2016)]

Various studies confirm that isoforms of COX-1 and COX-2 are critical for efficient viral replication. In one study Ray and Enquist showed that simultaneous inhibition of COX-1 and COX-2 caused a dramatic reduction of viral yield after HSV-1 infection [N. Ray and L. Enquist, J. Virol. 78, 3489-3501 (2004)]. Hill, et al., used microarrays to analyze gene expression in the trigeminal ganglion of mice infected with latent HSV-1, and found COX-2 gene expression significantly up regulated after reactivation [J. Hill, et al., Virus Genes 23, 273-280 (2001)]. Gebhardt reported that the selective COX-2 inhibitor celecoxib can suppress hyperthermic stress-induced herpes viral reactivation in the nervous system of mice [B. Gebhardt, et al., J. Ocul. Pharmacol. Ther. 21, 114-120 (2005)].

Functional somatic syndromes (FSS) may be defined as conditions “characterized by patterns of persistent bodily complaints for which adequate examination does not reveal sufficiently explanatory structural or other specified pathology” [P. Henningsen, et al. (2007) Lancet 369, 946-954]. A diverse number of conditions are commonly described as FSS, including: fibromyalgia, irritable bowel syndrome, chronic fatigue syndrome, premenstrual syndrome, non-ulcer dyspepsia, chronic pain, chronic pelvic pain, hypoglycemia, low back pain, sick building syndrome, Gulf War syndrome, tension headache, tempo-mandibular joint disorder, repetitive strain injury, multiple chemical sensitivity, interstitial cystitis, chronic Lyme disease, depression, post-traumatic stress disorder (PTSD), chronic anxiety disorder, food hypersensitivity, and brain fog or cognitive dysfunction.

Despite the broad range of FSS conditions, these disease states may have a common etiology rather than being distinct syndromes. Wessely and colleagues concluded on the basis of a literature review that there was substantial overlap between these conditions and that their similarities were greater than their differences, proposing the concept of a general functional somatic syndrome [S. Wessely, et al. (1999) Lancet 354, 936-939]. A common etiology for FSS was also explored by Bland, who pointed out that when the allostatic load, the combined external and internal stress, exceeds the ability of the patient to maintain allostasis, alterations in function occur giving rise to symptomatic FSS [J. Bland (2008) Alt. Therapies 14, 14-16.] Somatic syndrome disorder further classifies IBS, fibromyalgia, and ME/CFS and other conditions.

U.S. Pat. No. 8,809,351 provides additional support for the concept that Herpes virus is a common etiological stressor that gives rise to fibromyalgia and FSS. Briefly, U.S. Pat. No. 8,809,351 disclosed that of nineteen patients suffering from fibromyalgia, eighteen showed the presence of HSV-1 DNA. HSV-1 infection was confirmed through the detection of the virus protein ICP8 in biopsies of the positive patients. U.S. Pat. No. 8,809,351 further demonstrated that administration of the combination of famciclovir and celecoxib effectively treated fibromyalgia, brain fog, inflammatory bowel disease (IBS), chronic fatigue syndrome (CFS), chronic pain, and brain fog.

Herpes virus markedly decreases both natural killer (NK) and CD8+ T cell activity, both of which are critical components of the immune response to viral infections. In this way, Herpes virus may be an immunosuppressing infection. Downregulation of NK and CD8+ T cell activity has been shown in chronic pain, ME/CFS, and long COVID. Without being bound by theory, it is hypothesized that following an acute SARS-CoV2 infection, a subsequent Herpes virus infection (or a coinfection of Herpes virus and SARS-CoV2) persists as a nociceptive low-grade illness that results in long COVID. Given the similarity of symptoms of long COVID with ME/CFS, it is theorized that administration of an antiviral agent and a COX-2 inhibitor can be an effective treatment for long COVID.

### C. Pharmaceutical Compositions

The compounds of the present invention can be administered in a unit dosage from. If desired, multiple doses per day of the unit dosage form can be used to increase the total daily dose. Combinations of a COX-2 inhibitor and an antiviral compound are disclosed in U.S. Pat. No. 8,809,351, which is incorporated herein by reference. Particular embodiments include a combination of celecoxib and valacyclovir and a combination of celecoxib and famciclovir.

In another embodiment, there is provided a pharmaceutical composition, as described herein, wherein the amount of antiviral compound is present in a unit dosage form from about 250 mg to about 2000 mg.

In another embodiment, there is provided a pharmaceutical composition, as described herein, wherein the amount of antiviral compound is present in a unit dosage form from about 250 mg to about 1000 mg.

In another embodiment, there is provided a pharmaceutical composition, as described herein, wherein the amount of antiviral compound is present in a unit dosage form from about 250 mg to about 500 mg.

In another embodiment, there is provided a pharmaceutical composition, as described herein, wherein the antiviral compound is a guanine analog antiviral compound.

In another embodiment, there is provided a pharmaceutical composition, as described herein, wherein the antiviral compound is selected from the group consisting of famciclovir, valacyclovir, and acyclovir.

In another embodiment, there is provided a pharmaceutical composition, as described herein, wherein the antiviral compound is famciclovir.

In another embodiment, there is provided a pharmaceutical composition, as described herein, wherein the amount of famciclovir is present in a unit dosage form from about 250 mg to about 1000 mg.

In another embodiment, there is provided a pharmaceutical composition, as described herein, wherein the antiviral compound is valacyclovir.

In another embodiment, there is provided a pharmaceutical composition, as described herein, wherein the amount of valacyclovir is present in a unit dosage form from about 1000 mg to about 2000 mg.

In another embodiment, there is provided a pharmaceutical composition, as described herein, wherein the antiviral compound is acyclovir.

In another embodiment, there is provided a pharmaceutical composition, as described herein, wherein the amount of acyclovir is present in a unit dosage form from about 400 mg to about 1600 mg.

In another embodiment, there is provided a pharmaceutical composition, as described herein, wherein the amount of COX-2 inhibitor is present in a unit dosage form from about 7.5 mg to about 600 mg.

In another embodiment, there is provided a pharmaceutical composition, as described herein, wherein the amount of COX-2 inhibitor is present in a unit dosage form from about 15 mg to about 300 mg.

In another embodiment, there is provided a pharmaceutical composition, as described herein, wherein the amount of COX-2 inhibitor is present in a unit dosage form from about 50 mg to about 200 mg.

In another embodiment, there is provided a pharmaceutical composition, as described herein, wherein the COX-2 inhibitor is selected from the group consisting of celecoxib, meloxicam and a diclofenac—misoprostol combination.

In another embodiment, there is provided a pharmaceutical composition, as described herein, wherein the COX-2 inhibitor is celecoxib.

In another embodiment, there is provided a pharmaceutical composition, as described herein, wherein the amount of celecoxib is present in a unit dosage form from about 50 mg to about 600 mg.

In another embodiment, there is provided a pharmaceutical composition, as described herein, wherein the COX-2 inhibitor is meloxicam.

In another embodiment, there is provided a pharmaceutical composition, as described herein, wherein the amount of meloxicam is present in a unit dosage form from about 7.5 mg to about 15 mg.

In another embodiment, there is provided a pharmaceutical composition, as described herein, wherein the COX-2 inhibitor is a diclofenac—misoprostol combination.

In another embodiment, there is provided a pharmaceutical composition, as described herein, wherein the amount of diclofenac is present in a unit dosage form from about 50 mg to about 200 mg and the amount of misoprostol is present in a unit dosage form from about 200 μg to about 800 μg.

In one embodiment, there is provided a combination, comprising a therapeutically-effective amount of valacyclovir and a therapeutically-effective amount of celecoxib, wherein the amount of valacyclovir is present in a unit dosage form from about 750 mg to about 2000 mg, and wherein the amount of celecoxib is present in a unit dosage form from about 200 mg to about 800 mg.

In another embodiment, there is provided a combination, as described herein, wherein the amount of valacyclovir is present in a unit dosage form from about 750 mg to about 1050 mg.

In another embodiment, there is provided a combination, as described herein, wherein the amount of valacyclovir is present in a unit dosage form from about 1050 mg to about 1500 mg.

In another embodiment, there is provided a combination, as described herein, wherein the amount of valacyclovir is present in a unit dosage form selected from the group consisting of about 750 mg, about 1050 mg, about 1250 mg, about 1500 mg, and about 2000 mg.

In another embodiment, there is provided a combination, as described herein, wherein the amount of valacyclovir is present in a unit dosage form of about 750 mg or about 1250 mg.

In another embodiment, there is provided a combination, as described herein, wherein the amount of celecoxib is present in a unit dosage form from about 100 mg to about 400 mg.

In another embodiment, there is provided a combination, as described herein, wherein the amount of celecoxib is present in a unit dosage form from about 400 mg to about 800 mg.

In another embodiment, there is provided a combination, as described herein, wherein the amount of celecoxib is present in a unit dosage form selected from the group consisting of about 100 mg, about 200 mg, about 400 mg, and about 800 mg.

In another embodiment, there is provided a combination, as described herein, wherein the amount of celecoxib is present in a unit dosage form of about 200 mg or about 400 mg.

In another embodiment, there is provided a combination, as described herein, wherein the amount of valacyclovir is present in a unit dosage form of about 750 mg or about 1250 mg, and wherein the amount of celecoxib is present in a unit dosage form of about 200 mg or about 400 mg.

In one embodiment, there is provided a kit presentation, comprising a therapeutically-effective amount of valacyclovir or famciclovir in a first unit dosage form, and a therapeutically-effective amount of celecoxib, in a second unit dosage form, wherein the first and second unit dosage forms are separately enclosed in one or more containers, arranged in a single package or dispensing device, optionally comprising directions on how to use kit components suitable for administration to obtain a therapeutic outcome.

In another embodiment, there is provided a kit presentation, as described herein, wherein the amount of valacyclovir or famciclovir is present in a unit dosage form from about 750 mg to about 2000 mg, and wherein the amount of celecoxib is present in a unit dosage form from about 100 mg to about 800 mg.

In another embodiment, there is provided a dosage form wherein the amount of drug present may be from about 0.05% to about 95% by weight, more typically from about 2% to about 50% by weight of the dosage form.

For the treatment of the conditions referred to herein, the compounds described herein can be administered as described below.

### Oral Administration

The compounds of the present invention may be administered orally, including by swallowing, so that the compound enters the gastrointestinal tract, or absorbed into the blood stream directly from the mouth (e.g., buccal or sublingual administration).

Suitable compositions for oral administration include solid formulations such as tablets, lozenges and capsules, which can contain liquids, gels, or powders.

Compositions for oral administration may be formulated as immediate or modified release, including delayed or sustained release, optionally with enteric coating.

Liquid formulations can include solutions, syrups, and suspensions, which can be used in soft or hard capsules. Such formulations may include a pharmaceutically acceptable carrier, for example, water, ethanol, polyethylene glycol, cellulose, or an oil. The formulation may also include one or more emulsifying agents and/or suspending agents.

Tablets may contain a disintegrant, comprising from about 0.5% to about 35% by weight, more typically from about 2% to about 25% of the dosage form. Examples of disintegrants include methyl cellulose, sodium or calcium carboxymethyl cellulose, croscarmellose sodium, polyvinylpyrrolidone, hydroxypropyl cellulose, starch and the like.

Suitable lubricants, for use in a tablet, may be present in amounts from about 0.1% to about 5% by weight, and include calcium, zinc or magnesium stearate, sodium stearyl fumarate and the like.

Suitable binders, for use in a tablet, include gelatin, polyethylene glycol, sugars, gums, starch, hydroxypropyl cellulose and the like. Suitable diluents, for use in a tablet, include mannitol, xylitol, lactose, dextrose, sucrose, sorbitol and starch.

Suitable surface active agents and glidants, for use in a tablet, may be present in amounts from about 0.1% to about 3% by weight, and include polysorbate 80, sodium dodecyl sulfate, talc and silicon dioxide.

### Parenteral Administration

Compounds of the present invention may be administered directly into the blood stream, muscle, or internal organs. Suitable means for parenteral administration include intravenous, intra-muscular, subcutaneous intraarterial, intraperitoneal, intrathecal, intracranial, and the like. Suitable devices for parenteral administration include injectors (including needle and needle-free injectors) and infusion methods.

Compositions for parenteral administration may be formulated as immediate or modified release, including delayed or sustained release.

Most parenteral formulations are aqueous solutions containing excipients, including salts, buffering agents and carbohydrates.

Parenteral formulations may also be prepared in a dehydrated form (e.g., by lyophilization) or as sterile non-aqueous solutions. These formulations can be used with a suitable vehicle, such as sterile water. Solubility-enhancing agents may also be used in preparation of parenteral solutions.

### Topical Administration

Compounds of the present invention may be administered topically to the skin or transdermally. Formulations for this topical administration can include lotions, solutions, creams, gels, hydrogels, ointments, foams, implants, patches, and the like. Pharmaceutically acceptable carriers for topical administration formulations can include water, alcohol, mineral oil, glycerin, polyethylene glycol, and the like. Topical administration can also be performed by electroporation, iontophoresis, phonophoresis, and the like.

Compositions for topical administration may be formulated as immediate or modified release, including delayed or sustained release.

### Kits

Compound combinations of the present invention, wherein component A is an antiviral compound and component B is a COX-2 inhibitor as described herein, can be provided in a kit presentation, comprising an arrangement of components A and B in association with each other, as in a single package or in a drug dispensing device. Such kit presentations, used by a patient, may be dispensed by a hospital-formulary, retail pharmacist, or prescribing-physician.

In one example, the kit may comprise a single package, with therapeutically effective doses of components A and B, in the form of tablets or capsules in separate containers (e.g., bottles) held separately, as in a tray, and bound together in a single package using, for example, shrink wrap, tape, or a plastic or cardboard box enclosing the components.

In another example, separate, therapeutically-effective doses of combination components A and B, in the form of tablets or capsules, may be presented as co-packaged, in a single blister pack.

In another example, the kit presentation may provide therapeutically-effective doses of combination components A and B, in the form of tablets or capsules, which are co-dispensed from a device which delivers the components from a storage receptacle using, for example, one or more levers to co-dispense individual dose forms of components A and B to be administered in combination.

The kit presentation may also be used for parenteral administration of dose forms of Components A and B. For example, individual doses of components A and B in the form of lyophilized powders, either separately or with components A and B mixed together in therapeutically-effective doses, arranged in a package also comprising separately contained vials of sterile water or buffer solution, and optionally a sterile packaged syringe for administration of the dose combination following dissolution.

The kit presentation may further comprise directions, in compliance with approved instructions from a government agency (e.g., U.S. FDA), on how to use the kit components suitable for administration to obtain a therapeutic outcome.

### D. Methods of Treatment

The present disclosure further provides methods for treating a condition in a subject having or susceptible to having such a condition, by administering to the subject a therapeutically-effective amount of the compounds as described above. In one embodiment, the treatment is preventative treatment. In another embodiment, the treatment is palliative treatment. In another embodiment, the treatment is restorative treatment.

In one embodiment there is provided a method to treat a subject susceptible to or afflicted with a condition selected from the group consisting of fibromyalgia, chronic fatigue syndrome, irritable bowel syndrome, chronic pain, chronic headache, chronic neck pain, chronic back pain, chronic depression, chronic clinical anxiety disorder, post-traumatic stress disorder (PTSD), brain fog, cognitive dysfunction, and chronic interstitial cystitis, the method comprising administering to the subject a therapeutically-effective amount of an antiviral compound and a therapeutically-effective amount of a COX-2 inhibitor, in a dose weight ratio range from about one-to-one to about five hundred-to-one of the antiviral compound to the COX-2 inhibitor.

In one embodiment there is provided a method to treat a subject susceptible to or afflicted with Alzheimer's disease, the method comprising administering to the subject a therapeutically-effective amount of a COX-2 inhibitor, in a dose weight ratio range from about one-to-one to about five hundred-to-one of the antiviral compound to the COX-2 inhibitor. In a specific embodiment, the COX-2 inhibitor is celecoxib and the antiviral compound is famciclovir. In another specific embodiment, the COX-2 inhibitor is celecoxib and the antiviral compound is valacyclovir.

In one embodiment there is provided a method to treat a subject susceptible to or afflicted with long COVID, the method comprising administering to the subject a therapeutically-effective amount of a COX-2 inhibitor, in a dose weight ratio range from about one-to-one to about five hundred-to-one of the antiviral compound to the COX-2 inhibitor. In a specific embodiment, the COX-2 inhibitor is celecoxib and the antiviral compound is famciclovir. In another specific embodiment, the COX-2 inhibitor is celecoxib and the antiviral compound is valacyclovir.

In another embodiment, there is provided a method to treat a subject susceptible to or afflicted with a post-COVID19 illness including a post-acute infection syndrome (PAIS). In various embodiments, the method is used to treat a PAIS which is a sequelae of infection selected from the group consisting of SARS CoV-2 infection, Epstein-Barr virus infection, Ross River virus infection, human herpesvirus-6 infection, Varicella-zoster virus infection, Ebola virus infection, West Nile virus infection, Dengue virus infection, parvovirus infection, Borrelia burgdorferi infection, Coxiella burnetiid infection, and Mycoplasma pneumoniae infection. The method may comprise administering to the subject a therapeutically-effective amount of a COX-2 inhibitor, in a dose weight ratio range from about one-to-one to about five hundred-to-one of the antiviral compound to the COX-2 inhibitor. In a specific embodiment, the COX-2 inhibitor is celecoxib and the antiviral compound is famciclovir. In another specific embodiment, the COX-2 inhibitor is celecoxib and the antiviral compound is valacyclovir.

In another embodiment, there is provided a method to treat symptoms of orthostatic intolerance in a subject suffering from PAIS. In various embodiments, the method is used to treat a PAIS which is a sequelae of infection selected from the group consisting of SARS CoV-2 infection, Epstein-Barr virus infection, Ross River virus infection, human herpesvirus-6 infection, Varicella-zoster virus infection, Ebola virus infection, West Nile virus infection, Dengue virus infection, parvovirus infection, Borrelia burgdorferi infection, Coxiella burnetiid infection, and Mycoplasma pneumoniae infection. The symptoms may be dizziness, feeling faint, light-headedness, feeling about to blackout, blurred vision, tunnel vision, seeing spots, weakness, fatigue, trouble concentrating, head and neck discomfort, difficulty standing for a short time, difficulty standing for a long time, difficulty walking for a short time, and/or difficulty walking for a long time. The method may comprise administering to the subject a therapeutically-effective amount of a COX-2 inhibitor, in a dose weight ratio range from about one-to-one to about five hundred-to-one of the antiviral compound to the COX-2 inhibitor. In a specific embodiment, the COX-2 inhibitor is celecoxib and the antiviral compound is famciclovir. In another specific embodiment, the COX-2 inhibitor is celecoxib and the antiviral compound is valacyclovir.

In another embodiment, there is provided a method, as described herein, wherein the dose weight ratio range is from about one-to-one to about one hundred-to-one of the antiviral compound to the COX-2 inhibitor.

In another embodiment, there is provided a method, as described herein, wherein the dose weight ratio range is from about one-to-one to about fifty-to-one of the antiviral compound to the COX-2 inhibitor.

In another embodiment, there is provided a method, as described herein, wherein the dose weight ratio range is from about one-to-one to about twenty-to-one of the antiviral compound to the COX-2 inhibitor.

In another embodiment, there is provided a method, as described herein, wherein the dose weight ratio range is from about one-to-one to about five-to-one of the antiviral compound to the COX-2 inhibitor.

In another embodiment, there is provided a method, as described herein, wherein the amount of antiviral compound is present in a unit dosage form from about 250 mg to about 2000 mg.

In another embodiment, there is provided a method, as described herein, wherein the amount of antiviral compound is present in a unit dosage form from about 250 mg to about 1000 mg.

In another embodiment there is provided a method, as described herein, wherein the amount of antiviral compound is present in a unit dosage form from about 250 mg to about 500 mg.

In another embodiment, there is provided a method, as described herein, wherein the amount of famciclovir is present in a unit dosage form from about 250 mg to about 1000 mg.

In another embodiment, there is provided a method, as described herein, wherein the amount of valacyclovir is present in a unit dosage form from about 1000 mg to about 2000 mg.

In an alternative embodiment, there is provided a method, as described herein, wherein the antiviral compound is acyclovir.

In another alternative embodiment, there is provided a method, as described herein, wherein the amount of acyclovir is present in a unit dosage form from about 400 mg to about 1600 mg.

In another embodiment, there is provided a method, as described herein, wherein the amount of COX-2 inhibitor is present in a unit dosage form from about 7.5 mg to about 600 mg.

In another embodiment, there is provided a method, as described herein, wherein the amount of COX-2 inhibitor is present in a unit dosage form from about 15 mg to about 300 mg.

In another embodiment, there is provided a method, as described herein, wherein the amount of COX-2 inhibitor is present in a unit dosage form from about 50 mg to about 200 mg.

In an alternative embodiment, there is provided a method, as described herein, wherein the COX-2 inhibitor is meloxicam or a diclofenac—misoprostol combination.

In another embodiment, there is provided a method, as described herein, wherein the amount of celecoxib is present in a unit dosage form from about 50 mg to about 600 mg.

In another embodiment, there is provided a method, as described herein, wherein the COX-2 inhibitor is meloxicam.

In another embodiment, there is provided a method, as described herein, wherein the amount of meloxicam is present in a unit dosage form from about 7.5 mg to about 15 mg.

In another embodiment, there is provided a method, as described herein, wherein the COX-2 inhibitor is a diclofenac—misoprostol combination.

In another embodiment, there is provided a method, as described herein, wherein the amount of diclofenac is present in a unit dosage form from about 50 mg to about 200 mg and the amount of misoprostol is present in a unit dosage form from about 200 μg to about 800 μg.

In one embodiment, there is provided a method to treat a subject susceptible to or afflicted with Alzheimer's disease, the method comprising administering to the subject a therapeutically-effective combination of famciclovir and celecoxib, wherein the amount of famciclovir is administered in a total daily dose range from about 250 mg to about 1000 mg, and wherein the amount of celecoxib is administered in a total daily dose range from about 200 mg to about 800 mg, and wherein the combination administered produces no substantial adverse event.

In one embodiment, there is provided a method to treat a subject susceptible to or afflicted with Alzheimer's disease, the method comprising administering to the subject a therapeutically-effective combination of valacyclovir and celecoxib, wherein the amount of valacyclovir is administered in a total daily dose range from about 750 mg to about 2000 mg, and wherein the amount of celecoxib is administered in a total daily dose range from about 200 mg to about 800 mg, and wherein the combination administered produces no substantial adverse event.

In one embodiment, there is provided a method to treat a subject susceptible to or afflicted with long COVID, the method comprising administering to the subject a therapeutically-effective combination of famciclovir and celecoxib, wherein the amount of famciclovir is administered in a total daily dose range from about 250 mg to about 1000 mg, and wherein the amount of celecoxib is administered in a total daily dose range from about 200 mg to about 800 mg, and wherein the combination administered produces no substantial adverse event.

In one embodiment, there is provided a method to treat a subject susceptible to or afflicted with long COVID, the method comprising administering to the subject a therapeutically-effective combination of valacyclovir and celecoxib, wherein the amount of valacyclovir is administered in a total daily dose range from about 750 mg to about 2000 mg, and wherein the amount of celecoxib is administered in a total daily dose range from about 200 mg to about 800 mg, and wherein the combination administered produces no substantial adverse event.

In one embodiment, there is provided a method to treat a subject susceptible to or afflicted with PAIS, the method comprising administering to the subject a therapeutically-effective combination of famciclovir and celecoxib, wherein the amount of famciclovir is administered in a total daily dose range from about 250 mg to about 1000 mg, and wherein the amount of celecoxib is administered in a total daily dose range from about 200 mg to about 800 mg, and wherein the combination administered produces no substantial adverse event.

In one embodiment, there is provided a method to treat a subject susceptible to or afflicted with PAIS, the method comprising administering to the subject a therapeutically-effective combination of valacyclovir and celecoxib, wherein the amount of valacyclovir is administered in a total daily dose range from about 750 mg to about 2000 mg, and wherein the amount of celecoxib is administered in a total daily dose range from about 200 mg to about 800 mg, and wherein the combination administered produces no substantial adverse event.

In one embodiment, there is provided a method to treat symptoms of orthostatic intolerance in a subject suffering from PAIS, the method comprising administering to the subject a therapeutically-effective combination of valacyclovir and celecoxib, wherein the amount of valacyclovir is administered in a total daily dose range from about 750 mg to about 2000 mg, and wherein the amount of celecoxib is administered in a total daily dose range from about 200 mg to about 800 mg, and wherein the combination administered produces no substantial adverse event.

### E. Subjects

Suitable subjects to be treated according to the present invention include mammalian subjects. Mammals according to the present invention include, but are not limited to, human, canine, feline, bovine, caprine, equine, ovine, porcine, rodents, lagomorphs, primates, and the like, and encompass mammals in utero. Subjects may be of either gender and at any stage of development. In a particular embodiment, the subject is a human.

### F. Combinations and Combination Therapy

The compounds of the present invention, an antiviral compound and a COX-2 inhibitor, can be used as described herein or in combination with other pharmaceutically active compounds, to treat conditions such as those previously described above. The compounds of the present invention, an antiviral compound and a COX-2 inhibitor, and other pharmaceutically active compound(s) can be administered simultaneously (either in the same dosage form or in separate dosage forms) or sequentially. Accordingly, in one embodiment, the present invention comprises methods for treating a condition by administering to the subject therapeutically-effective amounts of the compounds of the present invention, an antiviral compound and a COX-2 inhibitor, and one or more additional pharmaceutically active compounds.

In another embodiment, there is provided a pharmaceutical composition comprising the compounds of the present invention, an antiviral compound and a COX-2 inhibitor, one or more additional pharmaceutically active compounds, and a pharmaceutically acceptable carrier.

In another embodiment, the one or more additional pharmaceutically active compounds are administered in any order or even simultaneously with the compounds of the present invention, an antiviral compound and a COX-2 inhibitor. If simultaneously, the multiple therapeutic agents are optionally provided in a single, unified form, or in multiple forms (by way of example only, either as a single pill or as two or more separate pills).

### EXAMPLES

The following example are merely illustrative, and do not limit this disclosure in any way.

### Example 1: Human Clinical Trials

### Objective:

To explore the efficacy of the combination of celecoxib and famciclovir in the treatment of patients diagnosed with myalgic encephalomyelitis/chronic fatigue syndrome (ME/CFS).

### Study Design:

Thirty-three patient diagnosed with ME/CFS were treated with valacyclovir (average dose 2264 mg/d) and celecoxib (400 mg/d).

### Patient Population and Diagnostic Criteria:

Adult men and women with a documented diagnosis of ME/CFS were selected. Screening assessment included a medical and psychological history and physical examination.

### ME/CFS

Center for Disease Control Criteria for CFS diagnosis requires three criteria:
i) The individual has had severe chronic fatigue for 6 or more consecutive months that is not due to ongoing exertion or other medical conditions associated with fatigue
ii) The fatigue significantly interferes with daily activities and work
iii) The individual concurrently has 4 or more of the following 8 symptoms:
   (a) post-exertion malaise lasting more than 24 hours
   (b) unrefreshing sleep
   (c) significant impairment of short-term memory or concentration
   (d) muscle pain
   (e) pain in the joints without swelling or redness
   (f) headaches of a new type, pattern, or severity
   (g) tender lymph nodes in the neck or armpit
   (h) a sore throat that is frequent or recurring

These symptoms should have persisted or recurred during 6 or more consecutive months of illness and they cannot have first appeared before the fatigue. (Chronic fatigue syndrome: General information. Centers for Disease Control and Prevention. www.cdc.gov/cfs/general)

### Cognitive Dysfunction or Impairment

Cognitive dysfunction or impairment, also referred to as brain fog or mental fog, is the loss of intellectual functions (such as thinking, remembering, and reasoning) of sufficient severity to interfere with daily functioning. Patients with cognitive dysfunction have trouble with verbal recall, basic arithmetic, and concentration.

Patient improvement was quantified using the Mental Clutter Scale (Leavitt, et al. (2011) Psychological Reports 109, 445-452).

### Fatigue

Fatigue is a feeling of a loss of energy, a strong desire to rest and sleep, and/or a feeling of being constantly overtired, which can interfere with normal daily activities. Fatigue can be assessed by a survey of patients receiving a combination of an antiviral and a COX-2 inhibitor, wherein the patients report the presence and severity of fatigue. Any improvement in fatigue is assessed by asking the patient first if fatigue was improved, and if so, to report the degree of improvement of their overall fatigue during the course of treatment.

### Results:

The results, presented in the tables below, shows the relative improvement in fatigue and cognitive function. The results are based on patients self-reported improvement in their symptoms of fatigue and cognition. Table 1 shows the results of the clinical trial. Table 2 shows the average dose and average improvement.

TABLE 1
Clinically - Treated Patient Responses

| | Valacyclovir | Celecoxib | length of | Reported % | |
|---|---|---|---|---|---|
| Patient | dose | dose | treatment | Improvement | |
| ID | (mg/d) | (mg/d) | (wks) | Fatigue | Cognition |
| LS | 2000 | 400 | 144 | 25 | 50 |
| DT | 3000 | 400 | 5 | 100 | 100 |
| TB | 3000 | 400 | 5 | 60 | 60 |
| IS | 2000 | 400 | 104 | 50 | 70 |
| PW | 2000 | 400 | 126 | 85 | 75 |
| RB | 2375 | 400 | 64.57 | 50 | 75 |
| KK | 2250 | 400 | 138 | 85 | 80 |
| CM | 2063 | 400 | 102.43 | 100 | 100 |
| JB | 2077 | 400 | 115.86 | 100 | 50 |
| SM | 3000 | 400 | 63.29 | 75 | 0 |
| BT | 2250 | 400 | 45 | 40 | 60 |
| SM | 2065 | 400 | 9.71 | 80 | 50 |
| DC | 2100 | 400 | 31.43 | 85 | 75 |
| SH | 2000 | 400 | 117 | 70 | 70 |
| KZ | 2214 | 400 | 35.86 | 10 | 30 |
| CH MO | 2000 | 400 | 58.71 | 30 | 30 |
| TB | 2077 | 400 | 144.86 | 65 | 60 |
| DT | 2106 | 400 | 114.86 | 80 | 100 |
| TB | 2058 | 400 | 31.14 | 60 | 60 |
| KW | 2000 | 400 | 151.29 | 80 | 75 |
| CR | 2000 | 400 | 115.14 | 40 | 0 |
| MA | 2500 | 400 | 52 | 95 | 95 |
| RB | 2000 | 400 | 321.29 | 95 | 98.5 |
| LB | 3000 | 400 | 12 | 95 | 97 |
| JB | 2500 | 400 | 14.71 | 50 | 50 |
| KC | 3000 | 400 | 3.43 | 72.5 | 62.5 |
| MF | 2000 | 400 | 42 | 85 | 80 |
| RB | 2038 | 400 | 18 | 20 | 20 |
| NG | 2000 | 400 | 169.43 | 99 | 100 |
| KM | 2038 | 400 | 179.57 | 87.5 | 100 |
| MB | 2000 | 400 | 145.43 | 30 | 30 |
| KZ | 3000 | 400 | 2.71 | 30 | 30 |
| SS | 2000 | 400 | 132.43 | 50 | 50 |

TABLE 2
Average Doses and Improvement

| Average | Average | Average | | |
|---|---|---|---|---|
| Valacyclovir | Celecoxib | Length of | | |
| dose | dose | Treatment | Average % Improvement | |
| (mg/d) | (mg/d) | (wks) | Fatigue | Cognition |
| 2264 | 400 | 85 | 66.0 | 63.1 |

### Example 2: Human Clinical Trial Protocol for Treatment of Long Covid

OBJECTIVE: To explore the safety and efficacy of the combination of an antiviral drug (famciclovir or valacyclovir)+celecoxib vs. placebo in the treatment of long COVID.

### Study Design:

An open-labeled, single-center, 14-week study was designed to explore the safety and efficacy of valacyclovir plus celecoxib for the treatment of prolonged symptoms caused by COVID-19 in adult female patients. The treatment consisted of daily doses of valacyclovir and celecoxib. Treated patients were dosed with 1.5 g valacyclovir and 200 mg celecoxib on a BID basis for 14 weeks in addition to the standard of care for treating long COVID. A comparison cohort of matching patients on standard of care was concurrently enrolled in which the comparison cohort received only standard of care to treat long COVID. The standard of care was current care provided by the center providers unless the long COVID patient was not a center patient. In that case, the standard of care was their ongoing care by PCP or another long COVID clinic.

Female patients were enrolled according to the inclusion criteria and assigned to treatment with either combination therapy (valcyclovir/celecoxib combination in addition to standard of care) (n=22) or placebo (standard of care alone) (n=17).

Qualified patients suffered primary long COVID (defined as experiencing one or more of fatigue, ongoing muscle weakness, reduced lung function, and below normal results in a 6-minute walking test [See Kuehn, JAMA, 325(11) (2021)], and an absence of other infections or other conditions that could compromise the interpretation of study results.

Patients underwent initial screening procedures, after which they proceeded with the washout of excluded medications, if required. Patients dependent upon opioids or narcotics for pain control were not enrolled in the study.

Due to the celecoxib component, patients in the treatment group discontinued regular use of all other non-steroidal anti-inflammatory drugs (NSAIDs) at the time of randomization. Acetaminophen was utilized throughout the study at doses not to exceed 3250 mg per day. Patients continued low-dose aspirin for cardioprotection (<325 mg/day), triptans and ergotamines for migraine, and dopaminergic agents for restless leg syndrome, as well as muscle relaxants, sleeping aids and benzodiazepines (assuming no evidence of abuse or dependency).

Metabolic profiles of each patient's concomitant medications were assessed to ensure that there is no risk of significant drug-drug interactions with either drug. For drugs that are metabolized by CYP2C9, the concomitant use of fluconazole—a potent CYP2C9 inhibitor—was avoided.

After ensuring that all entry criteria have been satisfied and washout successfully completed, patients returned for baseline assessments and randomization. The day of the baseline assessments will be referred to as Day 0; patients initiated study drug either with the evening dose on Day 0 or the morning dose on the following day (Day 1), continuing with BID treatment for the duration of the study.

### Inclusion Criteria:

1. Willing and able to read, understand, and sign the informed consent.
2. Female, 18-65 years of age, inclusive.
3. Each female patient must have a negative urine pregnancy test at Screening and Baseline unless she is post-menopausal.
4. Females of child-bearing potential must be willing to utilize an effective birth control method for the duration of their study participation.
5. Diagnosis of primary long COVID.
6. A urine drug screen performed at the Screening Visit must be negative for drugs of abuse such as methamphetamine, cocaine, phencyclidine (PCP), and non-disclosed amphetamines and opioids/opiates.
7. Qualified patients with mild to moderate depression should be clinically stable for three months, without risk of suicidal ideation or behavior. The dose of allowed antidepressants should have been stable for at least three months prior to screening.
8. In the opinion of the Investigator, the patient is willing and able to comply with all protocol-specified requirements.

### Exclusion Criteria:

1. Breastfeeding or pregnant.
2. Diagnosed with failed back syndrome, infectious arthritis, rheumatoid arthritis, systemic lupus erythematosus, or other systemic auto-immune diseases.
3. In the opinion of the Investigator, any clinically significant, uncontrolled or unstable medical, psychiatric or surgical condition that could affect the patient's ability to participate in the study or potentially compromise his/her well-being while enrolled in the study.
4. History of significant adverse reaction or allergy to study drugs.
5. History of suicide attempt or other suicidal behavior in the previous two years.
6. Any anticipated need for surgery that might confound results or interfere with the patient's ability to comply with the protocol.
7. Symptomatic and/or otherwise clinically significant cardiac disease.
8. Acute non-COVID systemic infection (e.g., HIV, hepatitis) or other active viral or bacterial infection during Screening or at the Baseline visit.
9. Currently receiving chronic systemic corticosteroids (>5 mg prednisone daily, or equivalent).
10. Uncontrolled sleep apnea. Patients successfully treated with CPAP or other devices are eligible.
11. Use of chronic nucleoside analog antiviral suppression therapy within one month of the Screening Visit, or requiring on average more than one acute treatment course every two months.
12. 11. Current use of celecoxib in combination with valacyclovir or famciclovir.
13. The patient has undergone a malabsorptive weight loss procedure (e.g., Roux-en-Y or other bypass procedure).
14. Severe IBS-C or colonic inertia as evidenced by seven or more days between bowel movements.
15. In the opinion of the Investigator, evidence of clinically significant laboratory abnormality(ies) based on the results of the screening laboratory assessments and/or medical history.

### Study Drugs:

The study drug was commercially available generic valacyclovir tablets and celecoxib capsules. At Baseline, a 45-day supply of study drug was dispensed to patients in the treatment arm, and a 30-day supply was dispensed at Weeks 6 and 10. Each dose of study drug contained one 200 mg celecoxib capsule and 1.5 grams of valacyclovir. Patients were instructed to take each dose twice daily (BID).

### Results:

### Efficacy Measures:

The primary outcome measure was change from baseline to Week 14 in the PROMIS fatigue 8a T-score

### Secondary Measures Will Include:

change from baseline to Week 6, 10, and 14 in the PROMIS fatigue 8a T-score;
change from baseline to Weeks 6, 12 and 14 in the self-reported NRS fatigue score;
change from baseline to Weeks 6, 10, and 14 in the self-reported average pain intensity score;
patient global status as measured on two PGIC scales at Weeks 6, 10, and 14;
change from baseline to Weeks 6, 10, and 14 in the HADS Anxiety domain and the HADS Depression domain; and
Change from baseline to Weeks 6, 10, and 14 in orthostatic intolerance testing.

### Safety Measures

Safety measures included vital signs (sitting blood pressure and heart rate, oral temperature, weight), adverse events and clinical laboratory assessments.

### Statistical Analyses:

The primary efficacy assessment for the determination of therapeutic efficacy was the change from baseline in the symptoms of long COVID. Change from baseline will be determined by comparing the baseline in the symptoms of long COVID to that determined at Weeks 6, 10, and 14.

The mean change from baseline in the combination drug treatment groups was compared to that determined for the placebo treatment group over 16 weeks of treatment using a mixed model repeated measures (MMRM). The null hypothesis was that there is no difference between treatment groups in terms of the mean change from baseline. Rejection of this hypothesis indicated efficacy of the combination therapy.

### Results

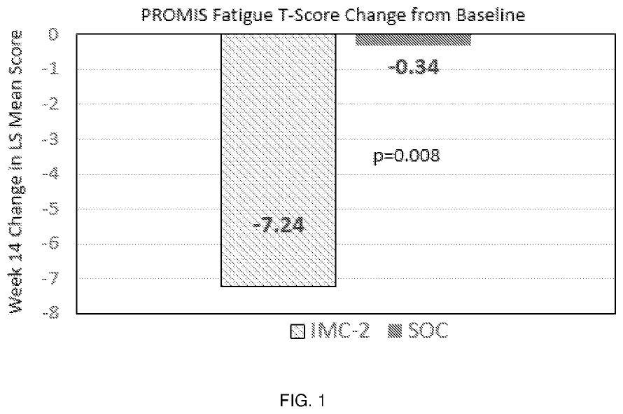

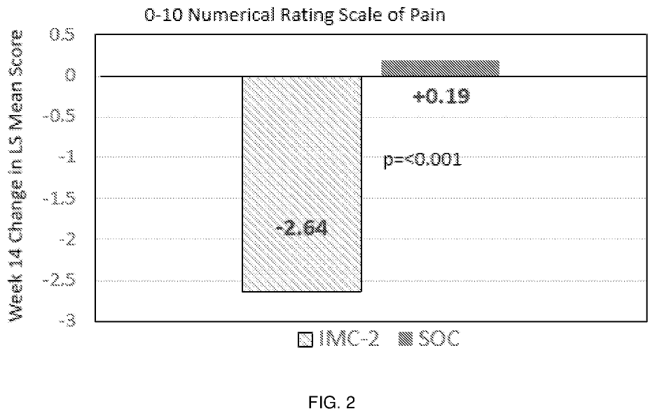

Tables 3 and 4 show the PROMIS fatigue score and NRS Fatigue score, respectively, in the subjects treated with the combination of 1.5 g valacyclovir and 200 mg celecoxib (represented as “IMC-2”). The “within group p value” column shows the statistical significance of the change in score as compared to baseline. The “comparative p value” column represents the statistical significance of the score between the treatment group (e.g., those patients that received the valacyclovir and celecoxib combination) with the standard of care (SOC) group (e.g., those patients that did not receive the valacyclovir and celecoxib combination). Addition of the valacyclovir/celecoxib combination to standard of care resulted in a statistically significant improvement in fatigue (as measured by both PROMIS and NRS scales) as compared to baseline. In contrast, subjects receiving only standard of care did not show a statistically significant improvement in fatigue as compared to baseline. Further, the results show a statistically significant improvement in most fatigue scores between the treatment group and control group. FIG. 1 shows the PROMIS fatigue T score change from baseline for both the combination and SOC control at Week 14. FIG. 2 shows the NRS fatigue score change from baseline for both the combination and SOC control at Week 14.

TABLE 3

| | | IMC-2 | | SOC |
|---|---|---|---|---|
| PROMIS | | change | | change |
| Fatigue | | from | | form |
| Score | IMC-2 score | baseline | SOC score | baseline |
| Baseline | 66.29 (n = 21) | | 66.53 (n = 17) | |
| Week 6 LS | 61.48* (n = 21) | −4.89 | 65.48 (n = 17) | −0.90*\hat{ } |
| mean score | | | | |
| Week 10 LS | 60.26* (n = 21) | −6.12 | No score taken | |
| mean score | | | | |
| Week 14 LS | 59.14* (n = 20) | −7.24 | 66.04 (n = 16) | −0.34*\hat{ } |
| mean score | | | | |

*statistical significance of p < 0.05 as compared baseline value of same treatment group
\hat{ }statistical significance of p < 0.05 between treatment group (IMC-2) and control group (SOC).

TABLE 4

| | | IMC-2 | | SOC |
|---|---|---|---|---|
| NRS Fatigue | | change | | change |
| Scale (0-10, 0 | | from | | form |
| is no fatigue) | IMC-2 score | baseline | SOC score | baseline |
| Baseline | 6.75 (n = 20) | | 6.94 (n = 17) | |
| Week 6 LS | 5.11* (n = 21) | −1.80 | 5.92* (n = 17) | −0.99 |
| mean score | | | | |
| Week 10 LS | 4.69* (n = 20) | −2.22 | 6.45 (n = 17) | −0.46\hat{ } |
| mean score | | | | |
| Week 14 LS | 4.45* (n = 20) | −2.64 | 7.10 (n = 16) | +0.19\hat{ } |
| mean score | | | | |

*statistical significance of p < 0.05 as compared baseline value of same treatment group
\hat{ }statistical significance of p < 0.05 between treatment group (IMC-2) and control group (SOC).

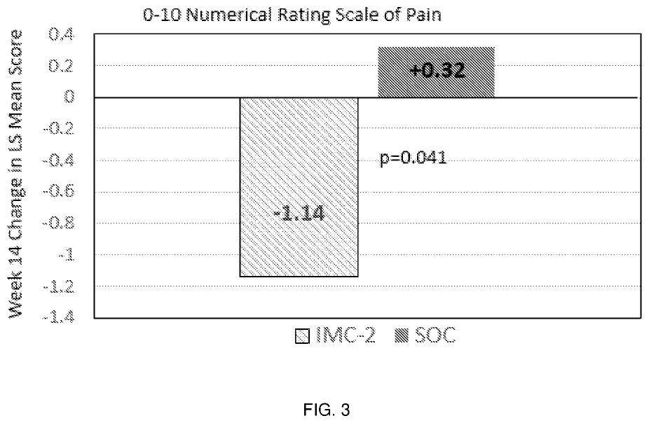

Table 5 shows the NRS pain scale in the subjects treated with the combination of 1.5 g valacyclovir and 200 mg celecoxib (represented as “IMC-2”). Addition of the valacyclovir/celecoxib combination to standard of care resulted in a statistically significant improvement in pain as compared to control subjects. FIG. 3 shows the NRS pain scale change from baseline for both the combination and SOC control at Week 14.

TABLE 5

| | | IMC-2 | | SOC |
|---|---|---|---|---|
| NRS Pain | | change | | change |
| Scale (0-10, 0 | | from | | form |
| is no pain) | IMC-2 score | baseline | SOC score | baseline |
| Baseline | 3.76 (n = 20) | | 4.56 (n = 17) | |
| Week 6 LS | 4.11* (n = 21) | −0.09 | 4.86 (n = 17) | +0.66 |
| mean score | | | | |
| Week 10 LS | 3.52* (n = 20) | −0.68 | 5.06 (n = 17) | +0.86\hat{ } |
| mean score | | | | |
| Week 14 LS | 3.06* (n = 20) | −1.14 | 4.51 (n = 16) | +0.31\hat{ } |
| mean score | | | | |

*statistical significance of p < 0.05 as compared baseline value of same treatment group
\hat{ }statistical significance of p < 0.05 between treatment group (IMC-2) and control group (SOC).

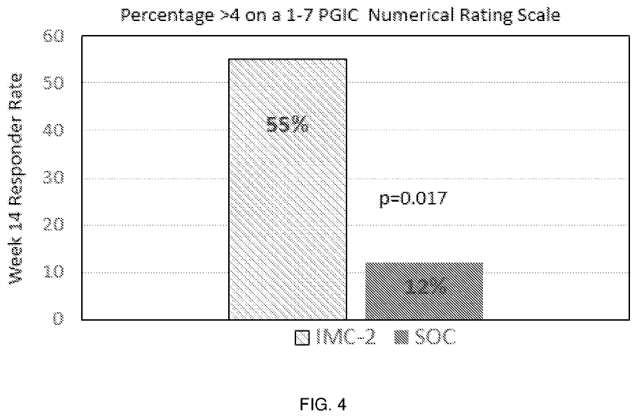

Tables 6 and 7 show the Patient Global Impression Change (PGIC) 1-7 scale and PGIC Improved 0-10 scale scores in the subjects treated with the combination of 1.5 g valacyclovir and 200 mg celecoxib (represented as “IMC-2”). Addition of the valacyclovir/celecoxib combination to standard of care resulted in a statistically significant improvement in PGIC and PGIC Improved as compared to control subjects. FIG. 4 shows the PGIC responder rate at Week 14. Tables 8 and 9 show the percentage of subjects reporting a “good result” according to PGIC and PGIC Improved. Here, a “good result” for the PGIC 1-7 scale refers to subjects that achieved a score of 5, 6, or 7 on the PGIC scale relative to their initial scores. A “good result” for the PGIC 0-10 scale refers to subject that achieves a score below 4 relative to their initial scores.

TABLE 6

| | | | Difference |
|---|---|---|---|
| | | | between |
| | IMC-2 | | IMC-2 and |
| PGIC (1-7, 7 is best) | score | SOC score | SOC groups |
| Week 6 LS mean score | 3.67 (n = 21) | 2.29 (n = 17) | 1.37\hat{ } |
| Week 10 LS mean score | 4.02 (n = 20) | Not measured | |
| Week 14 LS mean score | 3.93 (n = 20) | 2.53 (n = 17) | +1.4\hat{ } |

\hat{ }statistical significance of p < 0.05 between treatment group (IMC-2) and control group (SOC).

TABLE 7

| | | | Difference |
|---|---|---|---|
| | | | between |
| PGIC Improved (0-10, | IMC-2 | | IMC-2 and |
| 0 is best) | score | SOC score | SOC groups |
| Week 6 LS mean score | 4.00 (n = 21) | 5.75 (n = 17) | −1.75\hat{ } |
| Week 10 LS mean score | 3.48 (n = 20) | Not measured | |
| Week 14 LS mean score | 3.59 (n = 20) | 5.13 (n = 17) | −1.54\hat{ } |

\hat{ }statistical significance of p < 0.05 between treatment group (IMC-2) and control group (SOC).

TABLE 8

| | IMC-2 # | | | |
|---|---|---|---|---|
| | of “good | IMC-2 “good | SOC # of | SOC “good |
| PGIC | results” | result” rate | “good results” | result” rate |
| Week 6 | 10/21 | 45% | 3/17 | 18% |
| Week 10 | 10/21 | 48% | Not measured | |
| Week 14 | 11/20 | 55% | 2/11 | 12%^{#} |

^{#}statistical significance of Fisher Exact Score < 0.05 between treatment group (IMC-2) and control group (SOC).

TABLE 9

| | IMC-2 # | | | |
|---|---|---|---|---|
| PGIC | of “good | IMC-2 “good | SOC # of | SOC “good |
| Improved | results” | result” rate | “good results” | result” rate |
| Week 6 | 9/21 | 43% | 0/7 | 0%^{#} |
| Week 10 | 9/20 | 45% | Not measured | |
| Week 14 | 16/20 | 55% | 1/11 | 6%^{#} |

^{#}statistical significance of Fisher Exact Score < 0.05 between treatment group (IMC-2) and control group (SOC).

Tables 10 and 11 show the Hospital Anxiety and Depression Scale (HADS) depression and anxiety scores, respectively, in the subjects treated with the combination of 1.5 g valacyclovir and 200 mg celecoxib (represented as “IMC-2”). Addition of the valacyclovir/celecoxib combination to standard of care resulted in a statistically significant improvement in HADS depression score at Week 6 and in HADS Anxiety score at Week 14 as compared to control subjects.

TABLE 10

| | | IMC-2 | | SOC |
|---|---|---|---|---|
| | | change | | change |
| HADS | | from | | form |
| Depression | IMC-2 score | baseline | SOC score | baseline |
| Baseline | 8.90 (n = 21) | | 7.53 (n = 17) | |
| Week 6 LS | 5.75 (n = 21) | −2.64 | 8.84 (n = 17) | +0.45\hat{ } |
| mean score | | | | |
| Week 10 LS | 5.44 (n = 21) | −2.95 | Not measured | |
| mean score | | | | |
| Week 14 LS | 5.85 (n = 20) | −2.54 | 7.78 (n = 16) | −0.61 |
| mean score | | | | |

\hat{ }statistical significance of p < 0.05 between treatment group (IMC-2) and control group (SOC).

TABLE 11

| | | IMC-2 | | SOC |
|---|---|---|---|---|
| | | change | | change |
| | | from | | form |
| HADS Anxiety | IMC-2 score | baseline | SOC score | baseline |
| Baseline | 8.14 (n = 21) | | 6.47 (n = 17) | |
| Week 6 LS | 7.08 (n = 21) | −0.45 | 7.24 (n = 17) | −0.29 |
| mean score | | | | |
| Week 10 LS | 5.93 (n = 21) | −1.60 | Not measured | |
| mean score | | | | |
| Week 14 LS | 2.35 (n = 20) | −1.27 | 8.30 (n = 16) | +0.77\hat{ } |
| mean score | | | | |

\hat{ }statistical significance of p < 0.05 between treatment group (IMC-2) and control group (SOC).

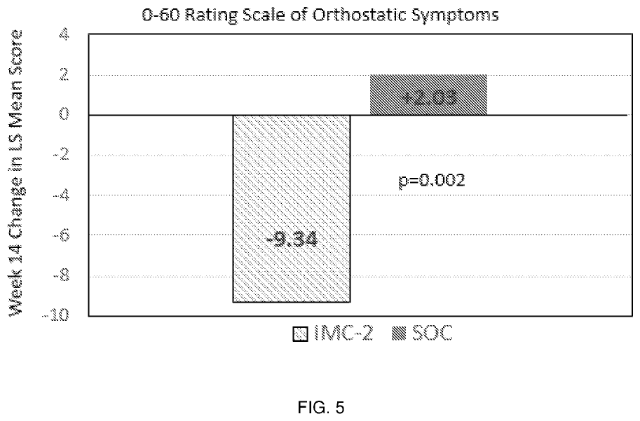

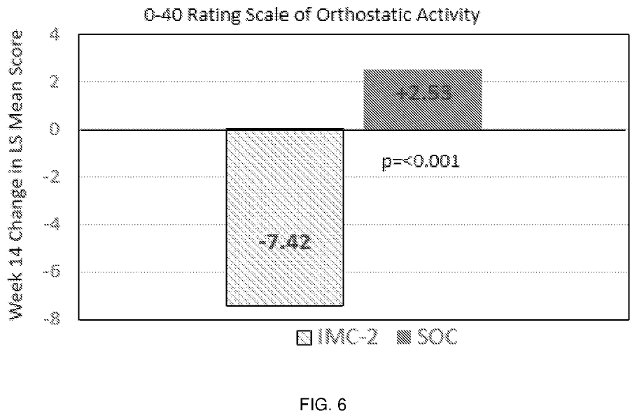

Tables 12 and 13 show the Orthostatic Intolerance Symptoms Assessment scale (OISA, OISAS) and Orthostatic Intolerance Daily Activity Impact Scale (IDAS), 5 respectively, in the subjects treated with the combination of 1.5 g valacyclovir and 200 mg celecoxib (represented as “IMC-2”). Addition of the valacyclovir/celecoxib combination to standard of care resulted in a statistically significant improvement in intolerance symptoms (as measured by OISA, OISAS) and daily activity (as measured by fIDAS) as compared to baseline. In contrast, subjects receiving only standard of care did not show a statistically significant improvement in either OISA, OISAS or OIDAS compared to baseline. Further, the results show a statistically significant improvement in OISA, OISAS and OIDAS scores between the treatment group and control group. FIG. 5 shows the OISA, OISAS change from baseline for both the combination and SOC control at Week 14. FIG. 6 shows the OIDAS change from baseline for both the combination and SOC control at Week 14.

TABLE 10

| | | IMC-2 | | SOC |
|---|---|---|---|---|
| | | change | | change |
| OISA, | | from | | form |
| OISAS | IMC-2 score | baseline | SOC score | baseline |
| Baseline | 35.76 (n = 21) | | 33.88 (n = 17) | |
| Week 6 LS | 27.78* (n = 21) | −7.76 | 36.62 (n = 17) | +1.94\hat{ } |
| mean score | | | | |
| Week 10 LS | 27.82* (n = 20) | −7.54 | Not measured | |
| mean score | | | | |
| Week 14 LS | 26.02* (n = 20) | −9.34 | 37.39 (n = 16) | +2.03\hat{ } |
| mean score | | | | |

*statistical significance of p < 0.05 as compared baseline value of same treatment group
\hat{ }statistical significance of p < 0.05 between treatment group (IMC-2) and control group (SOC).

TABLE 11

| | | IMC-2 | | SOC |
|---|---|---|---|---|
| | | change | | change |
| | | from | | form |
| OIDAS | IMC-2 score | baseline | SOC score | baseline |
| Baseline | 22.76 (n = 21) | | 25.06 (n = 17) | |
| Week 6 LS | 17.85* (n = 21) | −5.84* | 25.21 (n = 17) | +1.52\hat{ } |
| mean score | | | | |
| Week 10 LS | 17.55 (n = 20) | −6.14 | Not measured | |
| mean score | | | | |
| Week 14 LS | 16.27* (n = 19) | −7.42* | 26.04 (n = 17) | +2.53\hat{ } |
| mean score | | | | |

*statistical significance of p < 0.05 as compared baseline value of same treatment group
\hat{ }statistical significance of p < 0.05 between treatment group (IMC-2) and control group (SOC)

The results shown in Tables 3-11 and FIGS. 1-5 demonstrate that the combination of valacyclovir and celecoxib provides a statistically significant improvement over standard of care for long-COVID as measured by:
PROMIS fatigue at Weeks 6 and 14;
NRS fatigue scale at Weeks 10 and 14;
Global PGIC 1-7 scale continuous analysis at Weeks 6 and 14;
Global PGIC 1-7 scale responder analysis at Weeks 6 and 14;
Global PGIC 0-10 scale continuous analysis at Weeks 6 and 14;
Global PGIC 0-10 responder analysis at Weeks 6 and 14;
Orthostatic intolerance symptoms assessment (OISA) at Weeks 6 and 14;
Orthostatic intolerance daily activity impact (OIDAS) at Weeks 6 and 14;
NRS pain scale at Weeks 10 and 14; and
HADS anxiety and depression scale.

### Example 3: Treatment of Alzheimer's Disease

OBJECTIVE: To explore the efficacy of the combination of celecoxib and valacyclovir in the treatment of a patient diagnosed with Alzheimer's disease.

An adult 67-year old man presented with mild cognitive decline, including short- and long-term memory loss, muddled thinking and confusion, and difficulty with talking, reading, and organizing thoughts. The patient was prescribed 200 mg celecoxib b.i.d. and valacyclovir b.i.d. The valacyclovir dose was 1 g for 6 months, 1.5 g for months 6-9, and 2 g at 9 months. After 9 months of treatment, the patient completely recovered. Short- and long-term memory function returned to normal. The patient no longer experienced muddled thinking, confusion, or difficulty with talking, reading, or organizing thoughts.

### Example 4: Human Clinical Trial Protocol for Treatment of Alzheimer's Disease

OBJECTIVE: To explore the safety and efficacy of the combination of an antiviral drug (famciclovir or valacyclovir) and celecoxib vs. placebo in the treatment of Alzheimer's disease.

### Study Design:

Randomized, double-blind, placebo-controlled, 26-week study to evaluate the safety and efficacy of an antiviral drug (famciclovir or valacyclovir) and celecoxib combination for the treatment of patients suffering Alzheimer's disease. During the first week of treatment, a loading dose of the antiviral drug (famciclovir or valacyclovir) (2× maintenance dose) twice a day (BID) will be employed, followed by 25 weeks of maintenance dose of the antiviral drug (famciclovir or valacyclovir) BID. Depending on the patient population of the study group, use of a loading dose greater than 1000 mg/day is optional. The celecoxib dosage (also BID) will remain constant throughout the 26 weeks of active treatment.

Patients will be randomized to treatment with either combination therapy or placebo.

Qualified patients will have primary Alzheimer's (defined as experiencing one or more of accumulation of amyloid buildup, presence of mild cognitive impairment (including deficiencies of memory and/or other thinking problems), memory loss, word-finding difficulties, and visual/spatial problems. [See National Institutes of Aging Alzheimer's Disease Diagnostic Guidelines (www.nia.nih.gov/health/alzheimers-disease-diagnostic-guidelines)], and an absence of other conditions that could compromise the interpretation of study results.

Patients will undergo initial screening procedures, after which they proceed with the washout of excluded medications, if required. Patients dependent upon opioids or narcotics for pain control should not be enrolled in the study.

Due to the celecoxib component, patients will discontinue regular use of all other non-steroidal anti-inflammatory drugs (NSAIDs) at the time of randomization. Acetaminophen may be utilized throughout the study at doses not to exceed 3250 mg per day. Patients may also continue low-dose aspirin for cardioprotection (<325 mg/day), triptans and ergotamines for migraine, and dopaminergic agents for restless leg syndrome, as well as muscle relaxants, sleeping aids and benzodiazepines (assuming no evidence of abuse or dependency).

Metabolic profiles of each patient's concomitant medications should be assessed to ensure there is no risk of significant drug-drug interactions with either drug. For drugs that are metabolized by CYP2C9, the concomitant use of fluconazole—a potent CYP2C9 inhibitor—should be avoided.

After ensuring that all entry criteria have been satisfied and washout successfully completed, patients will return for baseline assessments and randomization. The day of the baseline assessments will be referred to as Day 0; patients will initiate study drug either with the evening dose on Day 0 or the morning dose on the following day (Day 1), continuing with BID treatment for the duration of the study.

Blood will be collected at the screening visit for safety assessments and for exploratory cytokine analyses (e.g., IL-1β, IL-4, IL-6, IL-8, IL-10, TNF-α, IFN-α, IFN-β, IFN-γ). A second sample for cytokine analyses will also be obtained at the Baseline/randomization visit. Follow-up blood sampling for safety and cytokine analyses will take place at the Week 8 and 16 visits (or at the time of early termination). A standard urinalysis panel will be included as part of the safety labs collected at screening at Week 8 and Week 16.

Study drugs will be over-encapsulated to maintain the double-blind, and active and placebo patients will receive identical-appearing supplies of study drug. Study drug will be provided in 2-week bottles; therefore, patients will receive 1, 2, or 3 separate bottles of each drug (or matching placebo) at every study visit, depending on the number of weeks until the next scheduled visit. For the first week only, the patient will receive a third bottle which contains additional antiviral drug (famciclovir or valacyclovir) to provide a one week loading dose. Patients will take one capsule BID (with meals) from each of the 3 bottles assigned for the first week, followed by one capsule BID from each of the 2 bottles provided for subsequent weeks. Patients will receive study drug treatment for a total of 21 weeks, with study visits during the active treatment phase of the study scheduled for Weeks 2, 8, 16, 22, and 26 or early termination.

### Inclusion Criteria:

1. Willing and able to read, understand, and sign the informed consent.
2. Male or female, 18-70 years of age, inclusive.
3. Each female patient must have a negative urine pregnancy test at Screening and Baseline unless she is post-menopausal.
4. Females of child-bearing potential must be willing to utilize an effective birth control method for the duration of their study participation.
5. Diagnosis of primary Alzheimer's disease.
6. In the opinion of the Investigator, the patient is willing and able to comply with all protocol-specified requirements.

### Exclusion Criteria:

1. Breastfeeding or pregnant.
2. Investigational drug usage within 30 days of Screening.
3. Diagnosed with failed back syndrome, infectious arthritis, rheumatoid arthritis, systemic lupus erythematosis, or other systemic auto-immune diseases.
4. In the opinion of the Investigator, any clinically significant, uncontrolled or unstable medical, psychiatric or surgical condition that could affect the patient's ability to participate in the study or potentially compromise his/her well-being while enrolled in the study.
5. Current systemic infection (e.g., HIV, hepatitis).
6. History of significant adverse reaction or allergy to study drugs.
7. In the opinion of the Investigator, evidence of clinically significant laboratory abnormality(ies) based on the results of the screening laboratory assessments and/or medical history.

### Study Drugs:

Each of the study drugs being evaluated in combination, as described herein, has been extensively studied in humans as well as animals. The doses and duration of treatment to be evaluated in these studies are consistent with each individual drug's current FDA-approved product labeling.

Study drug will be blinded by over-encapsulation of medications. Each medication will be provided in a separate bottle and clearly labeled in a blinded fashion. Placebo will be provided in capsules and bottles identical to those used for active study drug. All patients will take one capsule from each assigned bottle twice daily, with meals.

### Results:

### Efficacy Measures:

The primary outcome measure will be change from baseline to Week 26 in the ADAS-Cog score

### Secondary Measures Will Include:

change from baseline to Week 8, 16 and 22 in the ADAS-Cog score
change from baseline to Weeks 8, 16, 22 and 26 in the self-reported NRS fatigue score
change from baseline to Weeks 8, 16, 22 and 26 in the PROMIS sleep interference 8a T-score
proportion of patients with a PGIC rating of “very much improved” or “much improved” at Weeks 8, 16, 22 and 26

### Safety Measures

Safety measures include vital signs (sitting blood pressure and heart rate, oral temperature, weight), adverse events and clinical laboratory assessments.

### Statistical Analyses:

The primary efficacy assessment for the determination of therapeutic efficacy will be the change from baseline in the symptoms of Alzheimer's disease as measured by the ADAS-Cog assessment. Change from baseline will be determined by comparing the baseline in the symptoms of Alzheimer's disease to that determined at Weeks 8, 16, 22 and 26.

The mean change from baseline in the combination drug treatment groups will be compared to that determined for the placebo treatment group over 16 weeks of treatment using a mixed model repeated measures (MMRM). The null hypothesis will be that there is no difference between treatment groups in terms of the mean change from baseline. Rejection of this hypothesis will indicate efficacy of the combination therapy.

All mentioned documents are incorporated by reference as if herein written. When introducing elements of the present invention or the exemplary embodiment(s) thereof, the articles “a,” “an,” “the” and “said” are intended to mean that there are one or more of the elements. The terms “comprising,” “including” and “having” are intended to be inclusive and mean that there may be additional elements other than the listed elements. Although this invention has been described with respect to specific embodiments, the details of these embodiments are not to be construed as limitations.

1. A method to treat a subject susceptible to or afflicted with Alzheimer's disease, the method comprising:
administering to the subject a therapeutically effective amount of an antiviral compound and a therapeutically effective amount of a COX-2 inhibitor.

2. The method of claim 1, wherein the antiviral compound is famciclovir or valacyclovir.

3. The method of claim 1, wherein the COX-2 inhibitor is celecoxib.

4. The method of claim 1, wherein the antiviral compound is famciclovir and the COX-2 inhibitor is celecoxib.

5. The method of claim 4, wherein the amount of famciclovir is administered in a total daily dose range from about 750 mg to about 1500 mg, and wherein the amount of celecoxib is administered in a total daily dose range from about 200 mg to about 800 mg.

6. The method of claim 1, wherein the antiviral compound is valacyclovir and the COX-2 inhibitor is celecoxib.

7. The method of claim 6, wherein the amount of valacyclovir is administered in a total daily dose range from about 750 mg to about 2000 mg, and wherein the amount of celecoxib is administered in a total daily dose range from about 200 mg to about 800 mg.

8. A method to treat a subject susceptible to or afflicted with long COVID, the method comprising:
administering to the subject a therapeutically effective amount of an antiviral compound and a therapeutically effective amount of a COX-2 inhibitor.

9. The method of claim 8, wherein the antiviral compound is famciclovir or valacyclovir.

10. The method of claim 8, wherein the COX-2 inhibitor is celecoxib.

11. The method of claim 8, wherein the antiviral compound is famciclovir and the COX-2 inhibitor is celecoxib.

12. The method of claim 11, wherein the amount of famciclovir is administered in a total daily dose range from about 750 mg to about 1500 mg, and wherein the amount of celecoxib is administered in a total daily dose range from about 200 mg to about 800 mg.

13. The method of claim 8, wherein the antiviral compound is valacyclovir and the COX-2 inhibitor is celecoxib.

14. The method of claim 13, wherein the amount of valacyclovir is administered in a total daily dose range from about 750 mg to about 2000 mg, and wherein the amount of celecoxib is administered in a total daily dose range from about 200 mg to about 800 mg.

15. A method to treat a subject susceptible to or afflicted with a post-acute infection syndrome (PAIS) or treat symptoms of orthostatic intolerance in subjects with post-acute infection syndrome (PAIS), the method comprising:
administering to the subject a therapeutically effective amount of an antiviral compound and a therapeutically effective amount of a COX-2 inhibitor.

16. The method of claim 15, wherein the PAIS is a sequelae of infection selected from the group consisting of SARS CoV-2 infection, Epstein-Barr virus infection, Ross River virus infection, human herpesvirus-6 infection, Varicella-zoster virus infection, Ebola virus infection, West Nile virus infection, Dengue virus infection, parvovirus infection, Borrelia burgdorferi infection, Coxiella burnetiid infection, and Mycoplasma pneumoniae infection.

17. The method of claim 15, wherein the antiviral compound is famciclovir or valacyclovir.

18. The method of claim 15, wherein the COX-2 inhibitor is celecoxib.

19. The method of claim 15, wherein the antiviral compound is famciclovir and the COX-2 inhibitor is celecoxib.

20. The method of claim 19, wherein the amount of famciclovir is administered in a total daily dose range from about 750 mg to about 1500 mg, and wherein the amount of celecoxib is administered in a total daily dose range from about 200 mg to about 800 mg.

21. The method of claim 15, wherein the antiviral compound is valacyclovir and the COX-2 inhibitor is celecoxib.

22. The method of claim 21, wherein the amount of valacyclovir is administered in a total daily dose range from about 750 mg to about 2000 mg, and wherein the amount of celecoxib is administered in a total daily dose range from about 200 mg to about 800 mg.

23-32. (canceled)
